(19) United States
(12) Patent Application Publication
(10) Pub. No.: US 2014/0275887 A1
(43) Pub. Date: Sep. 18, 2014

(54) Systems And Methods For Monitoring Respiratory Depression

(71) Applicant: NELLCOR PURITAN BENNETT IRELAND, Mervue (IE)

(72) Inventors: Keith Batchelder, New York, NY (US); Scott McGonigle, Edinburgh (GB); James N. Watson, Dunfermline (GB); Andrew M. Cassidy, Edinburgh (GB); Paul S. Addison, Edinburgh (GB)

(21) Appl. No.: 13/832,928

(22) Filed: Mar. 15, 2013

(51) Int. Cl. A61B 5/00; A61B 5/0205; A61B 5/1455

(52) U.S. Cl. CPC A61B 5/7278; A61B 5/14551; A61B 5/0205; A61B 5/7282; A61B 5/7246; A61B 5/746; A61B 5/4836; A61B 5/742; A61B 5/0816
USPC 600/324

(57) ABSTRACT

Methods and systems are disclosed for analyzing a physiological respiratory signal in order to monitor respiratory depression events. In certain embodiments, respiratory depression is monitored by extracting a respiratory signal from a photoplethysmograph (“PPG”) signal, identifying a morphological characteristic of the respiratory signal, and generating a respiratory condition signal. In certain embodiments, an alarm and therapeutic intervention strategy are triggered upon determination of respiratory depression event. In certain embodiments, a plurality of physiological signals are used to determine a respiratory depression event

### SUMMARY OF THE DISCLOSURE

The present disclosure relates to physiological signal analysis and, more particularly, the present disclosure relates to analyzing a physiological respiratory signal in order to monitor respiratory depression events.

Respiratory depression, or hypoventilation, is a condition characterized by insufficient patient ventilation, and if left untreated can result in serious long-term consequences, including fatality. Among its causes, respiratory depression can arise from disease states, including stroke, asthma, pneumonia, or bronchitis. Additionally, a variety of different drugs, including opioids, benzodiazepines, barbiturates, gamma-hydroxybutyric acid, alcohol, and other sedatives can trigger dangerous episodes of respiratory depression. Respiratory depression may cause harmful changes in respiration rate, respiration effort, tidal volume, inspiration-expiration patterns, or other respiratory characteristics or combinations of respiratory characteristics. For example, respiratory rate or tidal volume may be reduced. Additionally, the rate of inspiratory flow may be reduced to a harmful level. These respiratory changes can increase systemic carbon dioxide levels due to insufficient gas exchange.

In certain cases, for example, when fentanyl is administered, respiratory depression is caused by out-of-phase movement between the chest and abdomen. This out-of-phase breathing can result in shortened inhalation and extended exhalation. In some cases, fentanyl has been reported to disproportionately increase both inspiration and expiration. For example, a patient may experience a 35% increase in inspiration and a 95% increase in expiration. These changes of respiration may alter the morphology of a respiratory signal.

Systems and methods disclosed herein use respiratory information from a photoplethysmograph (“PPG”) signal to monitor a patient for signs of respiratory depression. In certain embodiments, respiratory depression is monitored by extracting a respiratory signal from a PPG signal, identifying a morphological characteristic of the respiratory signal, and generating a respiratory condition signal. In certain embodiments, respiratory depression is monitored by detecting changes in the morphological characteristic over a plurality of time points. In certain embodiments, the detected change is indicative of a respiratory depression event. In certain embodiments, the detected change is predictive of the onset of respiratory depression, or may characterize the susceptibility of the patient to respiratory depression.

In certain embodiments, the morphological characteristic is compared to a database or look-up table to identify characteristics indicative of respiratory depression. The respiratory condition signal may be based on a quantitative scale, for example, where at least one of the high frequency or low frequency content of the respiratory signal is quantified. At least one of the inhalation periods, exhalation periods, or absence of inhalation-exhalation periods, or combination, may be quantified. One or more alarm and therapeutic intervention modes may be triggered based on the respiratory condition signal.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative patient monitoring system.

FIG. 2 is a block diagram of the illustrative patient monitoring system of FIG. 1 coupled to a patient.

FIGS. 3(a) and 3(b) show illustrative views of a scalogram derived from a PPG signal.

FIG. 3(c) shows an illustrative scalogram derived from a signal containing two pertinent components.

FIG. 3(d) shows an illustrative schematic of signals associated with a ridge of FIG. 3(c) and illustrative schematics of a further wavelet decomposition of derived signals.

FIGS. 3(e) and 3(f) are flow charts of illustrative steps involved in performing an inverse continuous wavelet transform.

FIG. 4 is a block diagram of an illustrative continuous wavelet processing system.

FIG. 5 shows an illustrative PPG signal obtained from a patient.

FIG. 6 depicts an illustrative respiratory signal with inhalation, exhalation, and intermediate periods.

FIG. 7 depicts an illustrative respiratory signal with representative inhalation and exhalation.

FIG. 8 depicts an illustrative respiratory signal.

FIG. 9 is a flow chart of illustrative process steps for determining physiological information from a physiological signal.

FIG. 10 is a flow chart of illustrative process steps for extracting respiratory characteristics with a scalogram.

FIG. 11 is a flow chart of illustrative process steps for identifying a breathing class.

FIG. 12 is a flow chart of illustrative process steps for generating a global marker for respiratory depression.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
I(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I₀−(sβ₀+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} , \qquad ( 3 )$$

3. Eq. 3, evaluated at the Red wavelength λ_{R}, is divided by Eq. 3 evaluated at the IR wavelength λₘ in accordance with

$$\frac{\frac{d log I ( {λ}_{R} )}{d t}}{\frac{d log I ( {λ}_{IR} )}{d t}} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R . \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{\frac{d I}{d t}}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R , \qquad ( 11 )$$

which defines a cluster of points whose slope of y versus x will give R when

x=[I(t₂,λ_{IR})−I(t₁,λ_{IR})]I/(t₁,λ_{R}), (12)

and

y=[I(t₂,λ_{R})−I(t₁,λ_{R})]I/(t₁,λ_{IR}), (13)

FIG. 1 is a perspective view of an embodiment of a patient monitoring system 10. In an embodiment, system 10 is implemented as part of a pulse oximetry system. System 10 may include a sensor 12 and a monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue. Sensor 12 or monitor 14 may further include, but are not limited to software modules that calculate respiration rate, airflow sensors (e.g., nasal thermistor), ventilators, chest straps, transthoracic sensors (e.g., sensors that measure transthoracic impedance).

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be a charged coupled device (CCD) sensor. In another embodiment, the sensor array may be made up of a combination of CMOS and CCD sensors. A CCD sensor may comprise a photoactive region and a transmission region for receiving and transmitting data whereas the CMOS sensor may be made up of an integrated circuit having an array of pixel sensors. Each pixel may have a photodetector and an active amplifier.

Emitter 16 and detector 18 may be on opposite sides of a digit such as a finger or toe, in which case the light that is emanating from the tissue has passed completely through the digit. Emitter 16 and detector 18 may be arranged so that light from emitter 16 penetrates the tissue and is reflected by the tissue into detector 18, such as a sensor designed to obtain pulse oximetry data from a patient's forehead.

The sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the effort or oximetry reading may be passed to monitor 14. As shown, monitor 14 includes a display 20 configured to display a patient's physiological parameters or information about the system. In the embodiment shown, monitor 14 also includes a speaker 22 to provide an audible sound that may be used in various other embodiments, such as sounding an audible alarm in the event that a patient's physiological parameters are not within a predefined normal range.

Sensor 12, or the sensor array, may be communicatively coupled to monitor 14 via a cable 24. However, in other embodiments, a wireless transmission device (not shown) or the like may be used instead of or in addition to cable 24.

In the illustrated embodiment, system 10 also includes a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 is configured to calculate physiological parameters and to provide a display 28 for information from monitor 14 and from other medical monitoring devices or systems (not shown). For example, multi-parameter patient monitor 26 is configured to display an estimate of a patient's blood oxygen saturation (referred to as an “SpO₂” measurement) generated by monitor 14, pulse rate information from monitor 14 and blood pressure from a blood pressure monitor (not shown) on display 28.

Monitor 14 is communicatively coupled to multi-parameter patient monitor 26 via a cable 32 or 34 that is coupled to a sensor input port or a digital communications port, respectively, and/or may communicate wirelessly (not shown). In addition, monitor 14 and/or multi-parameter patient monitor 26 may be coupled to a network to enable the sharing of information with servers or other workstations (not shown). Monitor 14 may be powered by a battery not shown) or by a conventional power source such as a wall outlet.

FIG. 2 is a block diagram of a patient monitoring system, such as patient monitoring system 10 of FIG. 1, which may be coupled to a patient 40 in accordance with an embodiment. Certain illustrative components of sensor 12 and monitor 14 are illustrated in FIG. 2. Sensor 12 includes emitter 16, detector 18, and encoder 42. In the embodiment shown, emitter 16 is configured to emit one or more wavelengths of light (e.g., Red and/or IR) into a patient's tissue 40. Hence, emitter 16 may include a Red light emitting light source such as Red light emitting diode (LED) 44 and/or an IR light emitting light source such as IR LED 46 for emitting light into the patient's tissue 40 at the wavelengths used to calculate the patient's physiological parameters. In one embodiment, the Red wavelength is between about 600 nm and about 700 nm, and the IR wavelength is between about 800 nm and about 1000 nm. In embodiments in which a sensor array is used in place of a single sensor, each sensor may be configured to emit a single wavelength. For example, a first sensor may emit only a Red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and any suitable wavelength of electromagnetic radiation may be appropriate for use with the present techniques. Detector 18 may be chosen to be specifically sensitive to the chosen targeted energy spectrum of the emitter 16.

In an embodiment, detector 18 is configured to detect the intensity of light at the Red and IR wavelengths. Alternatively, each sensor in the array may be configured to detect an intensity of a single wavelength. In operation, light enters detector 18 after passing through the patient's tissue 40. Detector 18 converts the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 sends the signal to monitor 14, where physiological parameters are calculated based on the absorption of the Red and IR wavelengths in the patient's tissue 40.

In an embodiment, encoder 42 may contain information about sensor 12, such as what type of sensor it is (e.g., whether the sensor is intended for placement on a forehead or digit) and the wavelength or wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 includes a memory on which one or more of the following information is stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 are transmitted to monitor 14. In the embodiment shown, monitor 14 includes a general-purpose microprocessor 48 connected to an internal bus 50. Microprocessor 48 is adapted to execute software, which may include an operating system and one or more applications, as part of performing the functions described herein. Also connected to bus 50 is a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 provides timing control signals to a light drive circuitry 60, which controls when emitter 16 is illuminated and multiplexed timing for the Red LED 44 and the IR LED 46. TPU 58 also controls the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In an embodiment, multiple separate parallel paths are provided having amplifier 66, filter 68, and A/D converter 70 for multiple light wavelengths or spectra received.

In an embodiment, microprocessor 48 determines the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. Such information may be stored in a suitable memory (e.g., RAM 54) and may allow monitor 14 to determine, for example, patient-specific threshold ranges in which the patient's physiological parameter measurements should fall and to enable or disable additional physiological parameter algorithms. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point at which a probe or sensor is attached.

Noise (e.g., from patient movement) can degrade a pulse oximetry signal relied upon by a physician without the physician's awareness. This is especially true if the monitoring of the patient is remote, the motion is too small to be observed, or the doctor is watching the instrument or other parts of the patient and not the sensor site. Processing physiological signals may involve operations that reduce the amount of noise present in the signals or otherwise identify noise components in order to prevent them from affecting measurements of physiological parameters derived from the physiological signals.

It will be understood that the present disclosure is applicable to any suitable signals and that PPG signals may be used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a physiological signal may be transformed using a continuous wavelet transform. Information derived from the transform of the physiological signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 14 )$$

where ψ*(t) is the complex conjugate of the wavelet function ψ(t), a is the dilation parameter of the wavelet and b is the location parameter of the wavelet. The transform given by Eq. 14 may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others, may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (15)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 16 )$$

and is useful for defining ridges in wavelet space when, for example, the Morlet wavelet is used. Ridges are defined as a locus of points of local maxima in the plane. A ridge associated with only the locus of points of local maxima in the plane is labeled a “maxima ridge.” Also included as a definition of a ridge are paths displaced from the locus of the local maxima. Any reasonable definition of a ridge may be employed in the methods described herein.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and a multiplication of the result by \sqrt{a}.

In the discussion of the technology which follows herein, the term “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram.”

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} , \qquad ( 17 )$$

where f_{c} is the characteristic frequency of the mother wavelet (i.e., at a=1) and becomes a scaling constant, and f is the representative or characteristic frequency for the wavelet at arbitrary scale a.

Any suitable wavelet function may be used in connection with the present disclosure. One of the most commonly used complex wavelets, the Morlet wavelet, is defined as

ψ(t)=π^{-1/4}(e^{i2πf}^{₀}^{t}−e^{-(2πf}^{₀}⁾^{²}²)e^{-t}^{²}^{/2}, (18)

where f₀ is the central frequency of the mother wavelet. The second term in the parentheses is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} . \qquad ( 19 )$$

This wavelet is a complex wave within a scaled Gaussian envelope. While both definitions of the Morlet wavelet are included herein, the function of Eq. 19 is not strictly a wavelet as it has a non-zero mean (i.e., the zero frequency term of its corresponding energy spectrum is non-zero). However, it will be recognized by those skilled in the art that Eq. 19 may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information.

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in Eq. 16, the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary, and may vary in scale, amplitude, or both, over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In an embodiment, a band ridge is defined as the locus of the peak values of these bands with respect to scale. For purposes of discussion, it may be assumed that band B contains the signal information of interest. Band B will be referred to as the “primary band.” In addition, it may be assumed that the system from which the signal originates, and from which the transform is subsequently derived, exhibits some form of coupling between the signal components in band A and band B. When noise or other erroneous features are present in the signal with similar spectral characteristics of the features of band B, then the information within band B can become ambiguous (i.e., obscured, fragmented or missing). In this case, the ridge of band A (referred to herein as “ridge A”) may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band Bin FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts, remove noise, combine bands, or any combination thereof. In one embodiment, there is an inverse continuous wavelet transform which allows the original signal to be recovered from its wavelet transform by integrating over all scales and locations, a and b, in accordance with

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} , \qquad ( 20 )$$

which may also be written as

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} . \qquad ( 21 )$$

where C_{g} is a scalar value known as the admissibility constant. It is wavelet-type dependent and may be calculated in accordance with

$${C}_{g} = {\int }_{0}^{∞} \frac{{\vert \hat{ψ} ( f ) \vert}^{2}}{f} d f . \qquad ( 22 )$$

FIG. 3(e) is a flow chart of illustrative steps that may be taken to perform an inverse continuous wavelet transform in accordance with the above discussion. An approximation to the inverse transform may be made by considering Eq. 20 to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

The present disclosure relates to physiological signal analysis and, more particularly, the present disclosure relates to analyzing a physiological respiratory signal in order to monitor respiratory depression events. In an embodiment, the above mentioned techniques are used to analyze physiological signals, such as a PPG signal, to monitor, measure, or predict respiratory depression events or susceptibility to respiratory depression events. For example, respiratory depression may be manifest in changes in respiratory rate, effort, tidal volume, or inhalation-exhalation patterns, or any combination thereof. As an additional example, respiratory depression may result in changes in a morphological characteristic of a respiratory signal derived from a PPG signal.

It will be understood that the present disclosure is applicable to any suitable signals and that PPG signals may be used merely for illustrative purposes. Those skilled in the art will recognize that the present disclosure has wide applicability to other respiration signals including, but not limited to carbon dioxide levels, oxygen saturation, transthoracic impedance, and air flow, or any other suitable signal or combination thereof. Those skilled in the art will recognize that the present disclosure has wide applicability to other biosignals including, but not limited to electrocardiogram, electroencephalogram, electrogastrogram, electromyogram, heart rate signals, pathological sounds, ultrasound, blood pressure, or any other suitable biosignal or any combination thereof. For example, the methods and systems disclosed herein may combine a plurality of signals, including PPG-based and non-PPG-based signals to generate a global marker for respiratory depression as described below.

FIG. 4 is a block diagram of an illustrative wavelet processing system in accordance with an embodiment. In an embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data signal generating equipment, or any combination thereof, to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals; astronomical signals, electrical signals, financial signals including financial indices, sound and speech signals, chemical signals, meteorological signals including climate signals, and/or any other suitable signal, and/or any combination thereof.

In an embodiment, signal 416 is coupled to processor 412, Processor 412 may be any suitable software, firmware, hardware, and/or combinations thereof, for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane, Any other suitable data structure may be used to store data representing a scalogram. The memory may be used by processor 412, to, for example, store any data related to any of the calculations described herein, including identifying morphological characteristics, identifying changes of morphological characteristics or signal patterns, and determining physiological information, such as respiratory depression events.

Processor 412 may be coupled to output 414. Output 414 may be any suitable output device such as one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 is implemented as part of sensor 12 and monitor 14, and processor 412 is implemented as part of monitor 14. It will be further understood that system 400 and/or system 10 may be adapted to derive from any other suitable signal sensed from a patient (i.e., patient 40) any other suitable physiological parameters, such as respiration rate and blood pressure. For example, microprocessor 48 may determine the patient's respiration rate and/or blood pressure using various algorithms and/or look-up tables based on the value of the received signals and/or data corresponding to the light received by detector 18.

After processor 412 represents the signals in a suitable fashion, processor 412 may then find and analyze selected features in the signal representation of signal 416 to determine respiratory depression events. Selected features may include the value, weighted value, or change in values with regard to energy, amplitude, frequency modulation, amplitude modulation, scale modulation, morphology, differences between features (e.g., distances between ridge amplitude peaks within a time-scale band), or any combination thereof. The selected features may be localized, repetitive, or continuous within one or more regions of the suitable domain space representation of signal 416. The selected features may not necessarily be localized in a band, but may potentially be present in any region within a signal representation. For example, the selected features may be localized, repetitive, or continuous in scale or time within a wavelet transform surface. A region of a particular size and shape may be used to analyze selected features in the domain space representation of signal 416. The region's size and shape may be selected based at least in part on the particular feature to be analyzed. As an illustrative example, in order to analyze respiratory depression, the region may be selected to have an upper and lower scale value in the time-scale domain such that the region covers a portion of the band, the entire band, or the entire band plus additional portions of the time-scale domain. The region may also have a selected time window width.

The systems and method described herein can be applied to monitor a patient for respiratory depression in a clinical setting. Embodiments of systems and methods for detecting respiratory depression events will now be discussed in connection with FIGS. 5-12.

In certain embodiments, processor 412 extracts a respiratory signal from a PPG using, for example, system 10 or system 400. FIG. 5 shows an illustrative signal 550 indicative of a PPG over time period t. Signal 550 may be of an oscillatory nature due to the patient's breathing (e.g., the baseline of signal 550 oscillates in relation to the patient's breathing as shown by baseline 560) and may include other oscillatory features, such as oscillatory pulses 555, that may be analyzed to identify respiratory depression events using the methods and systems described herein, Respiratory depression events may be derived from any suitable signal obtained using a sensor capable of measuring the respiration of a patient, such as patient 40 (FIG. 2). For example, a representative respiratory signal may be derived from a signal obtained from a flow meter or a chest band sensor. The signal may also be derived from other biosignals captured by one or more sensors of a suitable biosignal measurement system. For example, the signal may be derived from PPG signal data received from a pulse oximetry system such as pulse oximetry system 10 (FIG. 1), or from other biosignals including transthoracic impedance signals, capnograph signals, nasal thermistor signals, and/or electrocardiogram (EKG) signals. Although the techniques disclosed herein are described in terms of a respiration signal derived from a PPG signal, the disclosed techniques may be applied to other respiration signals or any other biosignals containing information indicative of respiratory depression events.

In some embodiments, a respiratory signal and one or more morphological characteristics are derived by obtaining a signal (e.g., PPG signal 450) from a sensor (e.g., oximeter 420) coupled to the patient, transforming the signal (e.g., using a continuous wavelet transform) to generate a primary scalogram from the wavelet transform as described above with respect to FIGS. 3(a) to 3(e), and analyzing a band of the primary scalogram (e.g., band B of FIG. 3(c)). For example, the scale or range of scales at which the band may appear on the primary scalogram is related to the frequency of the patient's breathing, or the patient's respiration rate.

A respiratory signal and at least one morphological characteristic may be determined by analyzing a ridge selected from the band of the primary scalogram (e.g., ridge B of band B in FIG. 3(c)), from a signal (e.g., PPG signal 550) or a portion of a signal. For example, the primary scalogram may contain ridges corresponding to, among others, the pulse ridge in the pulse scale-range. In some embodiments, only the primary scalogram is used to identify or detect ridges. Optionally, the primary scalogram may be used to compute one or more secondary scalograms after the pulse ridge loci are extracted. Any suitable identification method may be used to select ridges within the respiration scale range. An example technique is described in more detail in U.S. patent application Ser. No. 12/245,326 (Attorney Docket No. H-RM-01197 (COV-02)), filed Oct. 3, 2008, entitled “SYSTEMS AND METHODS FOR RIDGE SELECTION IN SCALOGRAMS OF SIGNALS,” which is incorporated by reference herein in its entirety. In some embodiments, these techniques may be used to determine respiratory effort, respiration rate, heart rate, or other physiological parameters of a patient affected by respiratory depression.

Changes in blood pressure may be due to changes in the interthoracic pressure during breathing, which may be indicative of lengthening or shortening of the exhalation period, changes in tidal volume, or changes in respiratory effort. These signals may used to identify or predict a respiratory depression event. In some embodiments, blood pressure may be measured using any continuous non-invasive blood pressure (“CNIBP”) approach, as more fully described in Chen et al. U.S. Pat. No. 6,599,251, entitled “CONTINUOUS NON-INVASIVE BLOOD PRESSURE MONITORING METHOD AND APPARATUS,” which is incorporated by reference herein in its entirety. For example, blood pressure may be measured invasively using an arterial line or may be measured non-invasively using a sphygmomanometer. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a patient's body.

In some embodiments, an individual probe or sensor (e.g., sensor 12) is used with a detector (e.g., detector 18) positioned anywhere suitable on patient 40 (i.e., in an area where a strong pulsatile flow may be detected, such as over arteries in the neck, wrist, thigh, ankle, ear, or any other suitable location) to detect a PPG signal for use with a CNIBP monitoring system or pulse oximeter. The PPG signal is then analyzed (e.g., using processor 412) and used to compute a time difference between two or more points in the detected PPG signal. From this time difference, blood pressure values may be computed on a continuous or periodic basis and used to identify or predict a respiratory depression event.

In certain embodiments, the processor 412 represents the signal 416 as having periods of inhalation and exhalation. In certain cases, as depicted in the respiratory signal 600 of FIG. 6, respiratory depression is manifested as shortened periods of inhalation 610 and exhalation 630 with intermediate periods 620 or pauses with no breathing between periods of inhalation 610 and exhalation 630. In certain cases respiratory depression is caused by out of phase movement between the chest and abdomen. This out of phase breathing can result in shortened inhalation and disproportionate, extended exhalation. For example, as depicted in FIG. 7, the respiratory signal 700 has short, quick inhalation peaks 710 followed by long, extended exhalation peaks 720.

In certain embodiments, respiratory depression (or an indication thereof) is identified from the respiratory signal by extracting information directly from the PPG signal and processing that information through one or more signal processing techniques. For example, FIG. 8 shows a representative respiratory signal 810 derived from the kurtosis of a PPG signal, such as PPG signal 550 from FIG. 5. As shown, peak 810 is higher and wider than peak 820, indicative of a change in breathing rate and relative depth of breathing. In addition, the morphology of the signal changes from peak 810 to peak 820. Peak 810 is not only larger than peak 820, but has a “shelf” 810a located on the descending side of the shelf, whereas the shelf is not found in the smaller peak 820. This change in the morphology of the signal at the two peaks is a respiratory condition that may be indicative of respiratory depression. Thus, the processor 412 classifies peaks 810 and 820 in terms of breathing rate, depth, and shape, for example, with a k^{th} nearest neighbor classifier algorithm, to generate a respiratory condition signal to identify respiratory depression events. Identifying the peak morphology may be important for respiratory signals where the depth of breathing cannot be identified quantitatively because the amplitude scale has arbitrary units, or units not related directly to depth of breathing. For example, the respiratory signal may be represented as a probability distribution or other transformed signal (the selected morphological characteristic may include features in a time-scale band in wavelet space or a resealed wavelet space as described above). Identifying and analyzing morphology and its changes in the signal can provide a more accurate depiction of respiratory depression indicators.

FIG. 9 is a flow chart of illustrative steps in a process 900 for determining respiratory depression events in accordance with an embodiment. The process 900 is performed by processor 412, but may be performed by any suitable processing device. For example, process 900 may be performed by a digital processing device, or implemented in analog hardware. It will be noted that the steps of the process 900 may be performed in any suitable order, and certain steps may be omitted entirely, as will be discussed in additional detail below.

At step 902, a signal is received from any suitable source (e.g., patient 40) using any suitable technique. The received signal 416 in step 902 is PPG signal 550 obtained from sensor 12 coupled to the patient 40. The PPG signal 550 is obtained from input signal generator 410, which includes an oximeter 420 coupled to sensor 418, to provide PPG signal 550 as signal 416. In an embodiment, the PPG signal has been stored in ROM 52, RAM 52, and/or QSM 72 (FIG. 2) in the past and may be accessed by microprocessor 48 within monitor 14 to be processed. Signal 416 may include one or more of a Red PPG signal component and an IR PPG signal component. The received signal may be based at least in part on past values of a signal, such as signal 416, which may be retrieved by processor 412 from a memory such as a buffer memory or RAM 54.

In certain embodiments, the process 900 is performed on an IR PPG signal component of a received signal, a Red PPG signal component of a received signal, or a combination thereof. A received signal may be generated by sensor unit 12, which may itself include any of the number of physiological sensors described herein. The received signal may include a plurality of signals, for example, a PPG signal and a blood pressure signal. The plurality of signals may be received in the form of a multi-dimensional vector signal or a frequency- or time-multiplexed signal. Additionally, the signal received at step 902 may be a derived signal generated internally to processor 412. Accordingly, the received signal may be a transformation of a signal 416, or may be a transformation of multiple such signals. For example, the received signal may be a ratio of two signals.

As shown, the process 900 advances to step 904 upon receiving the signal (e.g., PPG signal 550) at step 902. At step 904, at least one respiratory signal is extracted from the signal received in step 902. In an embodiment, the processor 412 performs a kurtosis analysis of the PPG signal 550 to extract the respiratory signal, for example, as shown by signal 800 in FIG. 8. In an embodiment of step 904, processor 412 derives the baseline 560 of the PPG signal 550, which baseline 560 is the respiratory signal. In an alternative embodiment, the respiratory signal is extracted by transforming the signal into any suitable domain, for example, a Fourier, wavelet, spectral, scale, time, time-spectral, time-scale, or any transform space. Other suitable respiratory signals may include respiratory rate, respiratory effort, tidal volume, and inhalation-exhalation signals.

After extracting a respiratory signal at step 904, the process 900 advances to step 906 where a morphological characteristic of the respiratory signal indicative of respiratory effort is identified. At step 906, the size, shape, and location of the one or more regions are adaptively manipulated using signal analysis techniques. The processing may be based at least in part on changing characteristics of the signal or changing features of the signal transformed into a domain space. The morphological characteristic may be determined based at least in part on analysis of the respiratory signal in another domain. For example, in certain embodiments, the selected characteristic includes quantifications of the frequency content of the respiratory signal. As an illustrative example, the morphology of the signal may be characterized by the frequency content of the signal, which may be indicative of respiratory depression event. For example, a ratio of the high frequency content of the respiratory signal to the low frequency content of the respiratory signal may be determined as an indicator of the patient respiratory condition or respiratory depression events. Any part of the frequency content, or any analysis involving frequency content or the frequency domain, may be used. Inhalation and exhalation magnitudes, periods, or other patterns may be indicative of a respiratory depression event of a patient. For example, as shown in FIG. 6, respiratory depression may be characterized by short inhalation periods 610 and exhalation periods 630 followed by a pause 620. Additionally, as shown in FIG. 7, respiratory depression may be characterized by brief inhalation 710 and extended exhalation 720. Respiratory depression events may be correlated with any of the above selected features, other suitable features, or any combination thereof.

In an alternative embodiment illustrated in FIG. 10, the respiratory signal is extracted and at least one morphological characteristic is identified with a scalogram. In a process 1000, a PPG signal (e.g., PPG signal 550) is received at step 1010 followed by a transformation of the PPG signal at step 1020 using a continuous wavelet transform. For example, processor 412 may transform the PPG signal using a continuous wavelet transform as described above using equation (14). At step 1030, a scalogram is generated in any suitable manner and based at least in part on the transformed signal from step 1020. For example, the scalogram may be generated using the energy density function equation (15) and may include some or all of the features described above with respect to FIGS. 3(a), 3(b), and 3(c). In an embodiment, the scalogram of the PPG signal includes any suitable number of bands containing pulse information and respiration information, and each band may include a ridge. The ridge may be continuous or may include any suitable number of ridge fragments.

The process 1000 advances to step 1040, where any suitable region of the scalogram is selected. For example, a portion of the scalogram containing a ridge fragment may be selected. The selection may be based upon a database of respiratory signals. The selection may also be based upon a change in the scalogram, for example, the selection may be compared to a scalogram retrieved by processor 412 from a memory device, such as ROM 52, RAM 54, an external memory device, or a remote device. Alternatively, the entire scalogram may be selected. The process 1000 may then advance to step 1050, where an identified respiratory characteristic is extracted from the selected scalogram portion. The technique may be applied by processor 412 or microprocessor 48 to at least a portion of the band corresponding to the selected ridge or at least a portion of the original scalogram. In an embodiment, processor 412 or microprocessor 48 includes any suitable software, firmware, or hardware, or combinations thereof for generating a sum along amplitudes vector and applying it to the selected region.

Referring back to FIG. 9, after identification of a morphological characteristic indicative of respiratory depression events (e.g. step 906, which may alternatively be performed at steps 1040 and 1050 of the process 1000), the identified characteristic is saved to a history at step 908. For example, the identified characteristic can be stored by processor 412 in RAM 54, an external memory device, or a remote device. At process step 910, a respiratory condition signal is generated. The respiratory condition signal may be qualitative or quantitative. In certain embodiments, the respiratory condition signal is a classification of the patient's breathing type. For example, the breathing may be classified as “normal” or “indicative of respiratory depression,” In certain embodiments, the classification is based upon a look-up table or database of respiratory conditions. In certain embodiments, the respiratory condition signal is generated by comparing at least a part of the signal to at least a part of the data within the saved history of step 908 to identify changes in the morphological characteristic. In certain embodiments, the respiratory condition signal may be based on Bayesian non-parametric classification models of the identified morphological characteristic of step 906. Processor 412 may dynamically interpret one or more morphological characteristics (e.g., peaks 810, 820) of a signal (e.g., signal 800) to generate a respiratory condition signal based upon models generated at least in part from patient data (e.g., signal 416) received throughout the monitoring session. In certain embodiments, the respiratory condition signal is generated from a clustering algorithm, for example, from a principal component analysis of the one or more identified morphological characteristics. In certain embodiments, the respiratory condition signal is a quantitative scale or confidence measure to indicate the severity of a respiratory depression event or the likelihood of a respiratory depression event. For example, the respiratory condition signal may be a scale of 1-10 (or any quantitative scale), where a low number indicates normal breathing and higher numbers indicate a more severe respiratory depression condition. By quantifying a confidence measure, the respiratory condition signal may also predict the likelihood of the onset of a respiratory depression event. In certain embodiments, the respiratory condition signal is a visual cue indicative of the patient's respiratory condition. For example, a green indicator may indicate a normal respiratory condition and a red indicator may indicate a state of respiratory depression. In certain embodiments, the respiratory condition signal is an audible tone or beeping with a pitch or frequency corresponding to different respiratory conditions, and specifically, to respiratory depression.

After the respiratory condition signal is generated at step 910, the systems and methods described herein may continue to monitor the patient and repeat or continue the steps 902-910. In certain embodiments, the process 900 proceeds to step 912, in which the respiratory condition signal is used to determine whether or not a respiratory depression event has occurred. This step may be accomplished by any suitable electronic, physical, or other means. For example, in certain embodiments, if a confidence measure meets a predetermined threshold, the patient is considered to be undergoing a respiratory depression event. When a respiratory depression event is identified, an alarm may be triggered at step 914. For example, the processor 412 may send an electronic event flag to a display, such as display 28, which would present an alarm signal. A graphical representation may be displayed in one, two, or more dimensions and may be fixed or change with time. A graphical representation may be further enhanced by changes in color, pattern, or any other visual representation. Alternatively, the alarm may be a specific audible tone, light color, pattern, or other suitable indicator. In certain embodiments, the alarm is an electronic communication signal within processor 412 or monitor 14.

In certain embodiments, the alarm triggered at step 914 triggers a therapeutic intervention strategy at step 916. The intervention strategy may be a predetermined event, for example, to adjust delivery of an IV-administered analgesic or other pharmacological agent. In certain embodiments, the intervention strategy may be a graphical representation or instructions shown on a display, such as display 28. The representation or instructions may be fixed or change with time. The intervention strategy may be automated or may be implemented partially or entirely by a care provider. In certain embodiments, the intervention strategy is a preset by a care provider and automatically implemented by processor 412 upon progression to step 916.

After or during the generation of a respiratory condition signal, the process 900 may begin again. Either a new signal may be received, or the physiological information determination may continue on another portion of the received signal(s). In an embodiment, processor 412 may continuously or periodically perform steps 902-916, or any subset or combination thereof, and update the physiological information. The process may repeat indefinitely, until there is a command to stop the monitoring and/or until some detected event occurs that is designated to halt the monitoring process. In an embodiment, processor 412 performs process 900 at a prompt from a care provider via user inputs 56. In an embodiment, processor 412 performs process 900 at intervals that change according to patient status. For example, process 900 will be performed more often when a patient is undergoing rapid changes in respiratory condition, and will be performed less often as the patient's condition stabilizes.

Several of the steps of the process 900 may be aided by the use of a predictive model to minimize the risks and dangerous effects of respiratory depression events. For example, a predictive model may be employed in at least one of step 906 for identifying a morphological characteristic, step 910 for generating a respiratory signal, step 912 for determining a respiratory depression event, step 914 for triggering an alarm, and step 916 for implementing a therapeutic intervention strategy. In an embodiment, a predictive computational model is based in part on at least one of the following data sources: the received signal (e.g., input signal 416); additional physiological signals; patient characteristics; historical data of the patient or other patients; and computational or statistical models of physiological processes. Processor 412 may retrieve any of these data sources from memory such as ROM 52 or RAM 54, from an external memory device, or from a remote device. The structure of a predictive computational model may, for example, be based on any of the following models: a neural network, a Bayesian classifier, and a clustering algorithm. In an embodiment, processor 412 develops a predictive neural network for identifying or predicting respiratory depression events based at least in part on historical data from the given patient and/or other patients. In some embodiments, processor 412 implements the predictive computational model as a hypothesis test. Processor 412 may continually refine or augment the predictive computational model as new patient data and/or physiological signals are received. The predictive model may also be refined based on feedback from the patient or care provider received through the user inputs 56. Other predictive frameworks may include rule-based systems and adaptive rule-based systems such as propositional logic, predicate calculus, modal logic, non-monotonic logic and fuzzy logic.

In certain embodiments, a plurality of analysis techniques may be used in combination to identify a breathing class. FIG. 11 is a flow chart of illustrative steps in a process 1100 that uses at least two analysis methods for identifying a breathing class. Process 1100 may be performed by processor 412, or may be performed by any suitable processing device. The steps of process 1100 may be performed in any suitable order, and certain steps may be omitted entirely.

The process 1100 proceeds by receiving a PPG signal at step 1110 and extracting a respiratory signal from the PPG at step 1120. For example, the processor 412 may perform a kurtosis analysis of the PPG signal or derive a baseline (e.g., 560 of the PPG signal 550). In certain embodiments, a plurality of respiratory signals are extracted. Process 1100 proceeds to step 1130 to derive respiratory characteristics by transform methods and/or to step 1150 to describe a breath morphology. In certain embodiments, the PPG signal received at step 1110 is the respiratory signal used at steps 1130 or 1150. In certain embodiments, step 1130 uses a first respiratory signal and step 1150 uses a second substantially different respiratory signal. Steps 1130 and 1150 may proceed in parallel or in series. In certain embodiments, only one of steps 1130 or 1150 may be performed unless a predetermined condition is met, such as adjusting a setting on monitor 20. For example, a care provider may choose to use both steps 1130 and 1150 to identify a breathing class, or may choose to use only one method step. In certain embodiments, the detection of a respiratory anomaly may trigger the use of either or both steps 1130 or 1150, for example, to provide improved monitoring.

At step 1130, at least one respiratory characteristic is derived by transform methods. For example, respiratory effort may be derived by continuous wavelet transform methods as described above. At step 1130, a respiratory characteristic may be derived by transform methods in any suitable domain, for example, a Fourier, wavelet, spectral, scale, time, time-spectral, or time-scale transform. In certain embodiments, respiratory characteristics derived at step 1130 include at least one of respiratory rate, respiratory effort, tidal volume, or inhalation-exhalation signals. The process 1100 proceeds to step 1140 where the at least one derived respiratory characteristic is saved to a history (e.g., RAM 54, an external memory device, or a remote device). In certain embodiments, a “First In, First Out” (FIFO) trend buffer is used to record data over a rolling time period. For example, the patient data from the previous 24 hours may be saved to the history.

At step 1150, the breathing morphology is described. In certain embodiments, peaks of the respiratory signal, such as peaks 810 and 820 of signal 800, are classified based on the respective peak morphologies. In certain embodiments, the morphology is described using adaptive machine learning techniques. For example, a k^{th} nearest neighbor classification algorithm may be used to classify the signal morphology. The classification may be based on previously stored data from the monitored patient or other patients, or it may be based on simulated morphology data. In other embodiments, the morphology is described by the frequency content, or ratio of frequencies. The process 1100 proceeds to step 1160 where the breath morphology is saved to a history (e.g., RAM 54, an external memory device, or a remote device). In certain embodiments, a “First In, First Out” (FIFO) trend buffer is used to record data over a rolling time period. For example, the patient data from the previous 24 hours may be saved to the history.

The process 1100 proceeds to step 1170 where a breathing class is identified. For example, the breathing may be classified as “normal” or “indicative of respiratory depression.” Other classifications may also be used to indicate one or more respiratory conditions. The breathing class may allow a care provider to rapidly assess the condition of a patient. In certain embodiments, Step 1170 combines the respiratory characteristic of step 1130 and the breath morphology of step 1150 to identify the breathing class. In certain embodiments, the respiratory characteristic and breath morphology may be converted to numeric values and combined to calculate a confidence measure to indicate the severity of a respiratory condition or the likelihood of a respiratory condition. For example, the values may be stored in a stochastic matrix used to calculate probability vectors of one or more defined states, such as respiratory depression. In certain embodiments, a probability density function is estimated for one or more respiratory conditions. In certain embodiments, a weighted average of the values is calculated over a predetermined time window (e.g., most recent 10 seconds). In certain embodiments, step 1170 is accomplished by Bayesian non-parametric classification models of the derived characteristic of step 1130 and the breath morphology of step 1150. In certain embodiments, values derived at steps 1130 and 1150 are used to estimate the probability of a respiratory depression event using other non-parametric models, such as a kernel density estimation. In certain embodiments, other suitable classification methods may be used, for example, non-parametric Bayesian estimates, neural networks, or any suitable heteroassociative function estimation method. Additional or alternative classification techniques may include rule based systems and adaptive rule based systems, such as propositional logic, predicate calculus, or modal, non-monotonic, or fuzzy logics.

If respiratory depression or any other respiratory anomaly is detected at step 1170, an alarm is triggered at step 1180. An alarm may also be triggered at step 1180 if an anomaly is identified at step 1140 or step 1160. For example, if the patient's respiratory rate reaches a predetermined minimum or maximum threshold, process 1100 may proceed to step 1180 to trigger an alarm even without completing step 1170.

In certain embodiments, non-PPG signals are used to determine respiratory depression events. Non-PPG signals may include, for example, transthoracic impedance, airflow, tidal volume, blood pressure, chest and abdomen motion, electrocardiogram, electromyogram, electroencephalogram, and pulse rate. These alternative physiological signals may be used independent of or, as shown in process 1200 depicted in FIG. 12, in combination with PPG signals. Process step 1210 for identifying PPG-based markers for respiratory depression may proceed in a substantially similar manner as steps 902-906 of process 900. At process step 1220 for identifying non-PPG-based markers for respiratory depression, a non-PPG signal is received. In certain embodiments, the non-PPG signal is the marker. In certain embodiments, the non-PPG signal is transformed and processed using techniques described above in relation to PPG signals. For example, the non-PPG signal may be transformed into a wavelet transform domain or other suitable domain. Step 1220 may use any suitable signal processing techniques. At step 1230, the PPG-based and non-PPG-based markers are used in combination to detect respiration patterns. In certain embodiments of step 1230, statistical modeling techniques, such as Bayesian nonparathetric models, are developed for a plurality of signals comprising both PPG and non-PPG signals and implemented by processor 412 to detect respiration patterns. For example, the combination of heart rate and inhalation-exhalation peaks may provide an increased confidence measure over either signal alone. At step 1240, a global marker for respiratory depression is generated using at least one of the detected respiration patterns of step 1230, The global marker for respiratory depression may be analogous to the respiratory condition signal generated at step 910, but is generated with a plurality of biosignals, for example, by processor 412. The global marker for respiratory depression may be qualitative or quantitative. In certain embodiments, the global marker for respiratory depression is a classification of the patient's breathing type (e.g., “normal” or “respiratory depression”). In certain embodiments, the global marker for respiratory depression is based upon a look-up table or database of respiratory conditions. In certain embodiments, the global marker for respiratory depression is generated by a comparative analysis of saved data, or dynamic classification models. In certain embodiments, the global marker for respiratory depression is generated from a clustering algorithm, for example, from a principal component analysis. In certain embodiments, the global marker for respiratory depression is a quantitative scale or confidence measure to indicate the severity of a respiratory condition or the likelihood of a respiratory condition. In certain embodiments, the global marker for respiratory depression is a visual or audio cue. The global marker for respiratory depression may be used in other processes, for example to trigger an alarm or a therapeutic intervention strategy similar to those described for process steps 912, 914, and 916.

The systems and methods described herein may have applications in other clinical applications. For example, in certain embodiments, monitoring respiratory depression may be used to assess the level of consciousness of a sedated patient. In certain embodiments, monitoring respiratory depression may be used to regulate patient-controlled analgesia or dosage levels of other administered pharmacological agents. In certain embodiments, monitoring respiratory depression may be used to assess apnea events. In certain embodiments, monitoring respiratory depression may be used to monitor stroke, asthma, pneumonia, bronchitis, or other disease states. In certain embodiments, monitoring respiratory depression may be used to monitory the efficacy of a therapy.

It will also be understood that the above method may be implemented using any human-readable or machine-readable instructions on any suitable system or apparatus, such as those described herein.

The foregoing is merely illustrative of the principles of this disclosure and various modifications can be made by those skilled in the art without departing from the scope and spirit of the disclosure. Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application.

The following claims describe various aspects of this disclosure.

1. A method for monitoring respiratory depression in a patient comprising:
receiving a photoplethysmograph (“PPG”) signal;
extracting, using processing equipment, a respiratory signal from the PPG signal;
identifying, using the processing equipment, a morphological characteristic of the respiratory signal; and
generating a respiratory condition signal indicative of the patient's breathing pattern based at least in part on the identified morphological characteristic.

2. The method of claim 1, further comprising detecting a change in the identified morphological characteristic.

3. The method of claim 2, further comprising at least one of determining a respiratory depression event based at least in part on the detected change in the identified morphological characteristic, predicting an onset of respiratory depression based at least in part on the detected change in the identified morphological characteristic, and characterizing a susceptibility of the patient to respiratory depression based at least in part on the detected change in the identified morphological characteristic.

4. The method of claim 3, wherein the PPG signal comprises a continuous signal.

5. The method of claim 3, wherein the PPG signal comprises a non-continuous signal.

6. The method of claim 3, wherein identifying the morphological characteristic is based at least in part on a k^{th} nearest neighbor classifier.

7. The method of claim 1, wherein generating the respiratory condition signal is based at least in part on a Bayesian non-parametric classifier of the identified morphological characteristic.

8. The method of claim 3, wherein generating the respiratory condition signal is based at least in part on comparing the identified morphological characteristic to a database of morphological characteristics.

9. The method of claim 3, wherein generating the respiratory condition signal is based at least in part on comparing the respiratory condition signal to a quantitative scale for respiratory condition signals.

10. The method of claim 3, further comprising, when the patient is sedated, determining a level of consciousness based at least in part on the identified morphological characteristic.

11. The method of claim 3, wherein the morphological characteristic of the respiratory signal is derived by a continuous wavelet transform.

12. The method of claim 3, further comprising deriving the respiratory rate, respiratory effort, tidal volume, or periods of inhalation and exhalation.

13. The method of claim 1, further comprising triggering an alarm based at least in part on the respiratory condition signal.

14. The method of claim 13, further comprising triggering a therapeutic intervention based at least in part on the respiratory condition signal.

15. The method of claim 3, wherein identifying the morphological characteristic comprises quantifying a high frequency content of the respiratory signal.

16. The method of claim 3, wherein identifying the morphological characteristic of the respiratory signal comprises quantifying a low frequency content of the respiratory signal.

17. The method of claim 3, wherein identifying the morphological characteristic of the respiratory signal comprises:
quantifying a high frequency content of the respiratory signal;
quantifying a low frequency content of the respiratory signal; and
computing a ratio of the high frequency content to the low frequency content.

18. The method of claim 3, wherein identifying the morphological characteristic of the respiratory signal comprises:
quantifying an inhalation period;
quantifying an exhalation period;
quantifying a period absent of inhalation and exhalation; and
computing a relationship between the periods of inhalation, exhalation, and absence of inhalation and exhalation.

19. The method of claim 3, further comprising:
selecting a plurality of markers of respiratory depression; and
generating a global marker of respiratory depression based at least in part on the plurality of markers of respiratory depression;

20. The method of claim 19, further comprising:
detecting a change pattern in at least one of the plurality of markers of respiratory depression; and
determining a respiratory depression event based at least in part on the detected change pattern.

21. The method of claim 20, wherein selecting a plurality of markers further comprises selecting a marker of respiratory depression from a patient monitor signal that is different from the PPG signal.

22. The method of claim 21, wherein selecting the plurality of markers of respiratory depression comprises selecting at least one of a signal indicative of respiratory rate, a signal indicative of respiratory effort, a characteristic of the morphology of a breathing signal, a metric based at least in part on an amplitude feature of a respiratory effort signal, a metric based at least in part on frequency content of the PPG signal, a measure of pulse wave velocity, and a measure of pulse wave pressure.
